(19) United States
(12) Patent Application Publication
(10) Pub. No.: US 2018/0228599 A1
(43) Pub. Date: Aug. 16, 2018

(54) TISSUE-DERIVED SCAFFOLDS FOR CORNEAL RECONSTRUCTION

(71) Applicant: THE JOHNS HOPKINS UNIVERSITY, Baltimore, MD (US)

(72) Inventors: Jennifer H. Elisseeff, Baltimore, MD (US); Jemin J. Chae, Baltimore, MD (US)

(21) Appl. No.: 15/750,689

(22) PCT Filed: Aug. 4, 2016
(86) PCT No.: PCT/US2016/045461

§ 371 Date: Feb. 6, 2018

Related U.S. Application Data
(60) Provisional application No. 62/202,033, filed on Aug. 6, 2015.

(51) Int. Cl. A61F 2/14; C12N 5/00; C12N 5/079; A61L 27/36

(52) U.S. Cl. CPC A61F 2/142; A61L 27/3604; C12N 5/0621; C12N 5/0068

(57) ABSTRACT

The present invention relates to methods for treating a corneal disease such as, for example, corneal blindness, or the refractive power of a cornea by generating a vitrified decellularized corneal inlay.

### REFERENCE TO RELATED APPLICATIONS

This application claims the benefit of U.S. Provisional Patent Application No. 62/202,033, filed on Aug. 6, 2015, which is hereby incorporated by reference for all purposes as if fully set forth herein.

### STATEMENT REGARDING FEDERALLY SPONSORED RESEARCH OR DEVELOPMENT

This invention was made with government support under grant no. W81XWH-09-2-0173 awarded by the National Institutes of Health. The government has certain rights in the invention.

### BACKGROUND OF THE INVENTION

Diseases affecting the cornea are a major cause of blindness worldwide, second only to cataract in overall importance. The epidemiology of corneal blindness is complicated and encompasses a wide variety of infectious and inflammatory eye diseases that cause corneal scarring, which ultimately leads to functional blindness. In addition, the prevalence of corneal disease varies from country to country and even from one population to another. For example, while cataract is responsible for nearly 20 million of the 45 million blind people in the world, the next major cause is trachoma which blinds 4.9 million individuals, mainly as a result of corneal scarring and vascularization. Ocular trauma and corneal ulceration are also significant causes of corneal blindness that are often underreported but may be responsible for as many as 1.5-2.0 million new cases of monocular blindness every year. Causes of childhood blindness (about 1.5 million worldwide with 5 million visually disabled) include xerophthalmia (350 000 cases annually), ophthalmia neonatorum, and less frequently seen ocular diseases such as herpes simplex virus infections and vernal keratoconjunctivitis.

Due to donor shortages, corneal blindness remains a significant clinical challenge, despite the fact that corneal transplantation has a high success rate. Accordingly, there is an urgent need to develop new corneal substitutes that have ideal characteristics, including transparency, proper concave shape, biocompatibility and good integration with host tissue is essential to address these challenges of current therapeutic strategies.

### SUMMARY OF THE INVENTION

The present invention provides compositions and methods for treating corneal disease. In particular, the present invention provides material that may be used for corneal transplantation. In particular, the translational material described herein may be used as a reliable corneal substitute, as well as a stable corneal inlay.

In one aspect, the present invention provides a method for treating a corneal disease in a subject that includes the steps of obtaining a tissue-derived scaffold, multiple decellularizing the tissue-derived scaffold, vitrifying the tissue-derived scaffold, cross-linking the tissue-derived scaffold, and generating a vitrified decellularized corneal inlay, thereby treating corneal disease in the subject.

In another aspect, the method further includes a step of molding the vitrified decellularized corneal inlay to produce a modified-shaped cornea for treatment in said subject.

In an embodiment, the vitrifying comprises controlled temperature and humidity.

In an embodiment, the temperature is between 4° C. to 37° C.

In an embodiment, the humidity is 40%.

In an embodiment, the cross-linking is riboflavin cross-linking.

In an embodiment, the vitrified decellularized corneal inlay is used to treat corneal blindness.

In an embodiment, the molded, vitrified decellularized corneal inlay is used to correct refractive error.

In an embodiment, the molding is performed using a 3D printer and 3D OCT (optical coherence tomography) system.

In an embodiment, the method further comprises addition of additives.

In an embodiment, the additives comprise small molecules.

In an embodiment, the small molecule is cylcodextrin.

In an embodiment, the tissue-derived scaffold is from a bladder.

In an embodiment, the transparency of the tissue-based scaffold is preserved.

### Definitions

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “modulate” is meant alter (increase or decrease). Such alterations are detected by standard art known methods such as those described herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration of at least 10%, 25%, 50%, 75%, or 100%.

As used herein, “obtaining” as in “obtaining an agent” includes synthesizing, purchasing, or otherwise acquiring the agent.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

Other features and advantages of the invention will be apparent to those skilled in the art from the following detailed description and claims.

### BRIEF DESCRIPTION OF THE DRAWINGS

FIGS. 1A-1B depict a cartoon and an image, respectively. FIG. 1A is a cartoon showing the anatomy of the cornea. FIG. 1B is an image showing the refractive power of the cornea.

FIG. 2 is an image of a patient showing the composition of a cornea, pre and post corneal transplant.

FIG. 3 is an image depicting different engineering approaches for corneal reconstruction, including synthetic approaches, tissue-based approaches, protein-based approaches, and self-assembly approaches.

FIGS. 4A-4D are images showing a schematic for lung tissue engineering, Petersen et al., Science 2010; 329: 538-541, incorporated herein by reference. FIG. 4A is an image showing a native adult rat lung that is cannulated in the pulmonary artery and trachea for infusion of decellularized solutions. FIG. 4B is an image showing acellular lung matrix devoid of cells after 2 to 3 hours of treatment. FIG. 4C is an image showing that after 4 to 8 days of culture, the engineered lung was removed from the bioreactor and was suitable for implantation. FIG. 4D is an image of the syngeneic rat recipient.

FIGS. 5A-5D are images showing decellularization, incorporated herein by reference Choi, et al., Ivest Opthalmol Vis Sci. 2011; 52: 6643-6650. FIG. 5C is an image showing a recipient that received decellularized posterior porcine stroma, showed a persistent epithelial defect for more than 3 weeks, and eventually the graft was rejected with severe edema and new vessels. FIG. 5D is a histological image showing a rejected graft that showed severe edema and inflammatory cellular infiltration extending from the periphery to the center.

FIGS. 6A-6D are images showing representative images of H&E stained sections of the decellularized porcine corneas, Sasaki, et al., Molecular Vision, 2009; 15:2022-2028, incorporated herein by reference. FIG. 6A is an image of a native cornea. FIG. 6B is an H&E stained section of a native cornea. FIG. 6C is an image of a cornea decellularized by UHP (ultrahigh hydrostatic pressure). FIG. 6D is an H&E stained section of cornea decellularized by UHP.

FIG. 7 is bar graph showing the results of a DNA assay in which immunogenic contents (e.g., debris) were removed.

FIG. 8 are images showing that the decellularized porcine cornea lost its transparency after procedures. Additionally, its concave shape was lost as well.

FIG. 9 is a Western blot with β-actin showing that the decellularized cornea (DC) included minimal cell contents that cause immunogenicity, S+T is SDS and triton-X treated cornea, scale bar is 100 μm.

FIGS. 10A-10B are bar graphs showing that ECM collagen and glycosaminoglycans were decreased following decellularizing procedures. FIG. 10A is a bar graph showing the OH-pro/dry weight (μg/mg) of native corneas and decellularized corneas, p<0.05. FIG. 10B is a bar graph showing the GAG (glycosaminoglycan)/dry weight (μg/mg) of native corneas and decellularized corneas, p<0.05.

FIG. 11 is an image showing a schematic of the method to produce vitrified cornea. After multiple decellularization (treated with 1% SDS and 1% Triton-X followed by 10% fetal bovine solution), the decellularized cornea underwent vitrification, followed up riboflavin crosslinking (using trephine). The treatment increased transparency of corneas. After the procedures, the transparency of the cornea was reconstructed microstructurally.

FIG. 12 is a graph showing the light transmittance of vitrified decellularized corneas. From 400 nm to 700 nm the percent transmittance was nearly 100% for native corneas, whereas for decellularized corneas, the percent transmittance was significantly less (e.g., approximately 40% at 400 nm).

FIGS. 13A-13B depict images showing the microstructure of a vitrified cornea. FIG. 13A is a transition electron microscopy image showing a native porcine cornea. FIG. 13B is a transition electron microscopy image showing a decellularized cornea. FIG. 13C is a transition electron microscopy image showing a vitrified decellularized cornea. After vitrification and crosslinking, the corneal structure was reconstructed.

FIGS. 14A-14 are images showing the microstructure of vitrified cornea. FIG. 14A is a transmission electron microscopy (TEM) image showing a sagittal view of the microstructure of a native porcine cornea. FIG. 14B is a TEM image showing a transversal view of the microstructure of a native porcine cornea. FIG. 14C is a TEM image showing a sagittal view of the microstructure of a decellularized cornea. FIG. 14D is a TEM image showing a transversal view of the microstructure of a vitrified decellularized cornea. FIG. 14E is a TEM image showing a sagittal view of the microstructure of a decellularized cornea. FIG. 14F is a TEM image showing a transversal view of the microstructure of a vitrified decellularized cornea. From the native corneas, to decellularized and vitrified corneas, the collagen fibers were thinning (FIGS. 14A, 14C, and 14E) and the density (FIGS. 14B, 14D, and 14F) changed. GAG loss was also expected. After decellularizing procedures, the cornea lost its native transparency and microstructure. With reconstructing vitrification and crosslinking procedures, the decellularized cornea was recovered to keep transparency and relatively organized collagen structure. The scale bar represents 100 nm.

FIGS. 15A-15B depict bar graphs showing the quantitative measurement of micro-structural changes. FIG. 15A is a bar graph showing the density of the collagen fiber (n/1 μm²) in native cornea, decellularized cornea and vitrified cornea. FIG. 15B is a bar graph showing the diameter of the collagen fiber (nm) in native cornea, decellularized cornea and vitrified cornea. The fiber density and diameter of the collagen were not fully reconstructed.

FIGS. 16A-16F depict images showing the macrostructure of vitrified cornea. FIG. 16A is an H&E stained image of a native porcine cornea. FIG. 16B is an image stained with Alcian blue for GAG of native porcine cornea. FIG. 16C is an H&E stained image of a decellularized cornea. FIG. 16D is an image stained with Alcian blue for GAG of decellularized cornea. FIG. 16E is an H&E stained image of a vitrified cornea. FIG. 16F is an image stained with Alcian blue for GAG of vitrified cornea. After vitrification and crosslinking, the corneal structure was partially reconstructed. Qualitatively, the GAG content was increased in the defined area after processing.

FIGS. 17A-17B show graphs showing the material stability of corneas. FIG. 17A is a bar graph showing the denature temperature of native cornea, decellularized cornea, and vitrified decellularized cornea, p<0.05. FIG. 17B is a graph showing the heat flow endo down (mW) versus temperature of native cornea, decellularized cornea, and vitrified decellularized cornea. The material stability changed following the processes but the denature temperature of the vitrified cornea was not significantly different with that of the native cornea. After vitrification and crosslinking, the material thermal stability of DC was increased as that of the native cornea.

FIGS. 18A-18B depict graphs showing results of mechanical tests for corneas. FIG. 18A is a bar graph showing compressive modulus (KPa) of native cornea, decellularized corneas, vitrified cornea and human cornea. Significance was established by ANOVA and Turkey's post-hoc test at p=0.05. FIG. 18B is a graph showing a suturability test of native cornea, decellularized corneas, vitrified cornea, and a suture. The elastic modulus of the vitrified cornea was similar to that of the native human cornea. The vitrified cornea was a suturable material.

FIG. 19 is a graph showing the degradation rate of corneas in collagenase type I solution. The degradation rate of DC was increased after vitrification and crosslinking processes compared to that of native cornea. The vitrified cornea has potential to integrate with the host tissue.

FIGS. 20A-20D are immunocytochemistry images showing that dead and live cell analysis indicated that vitrified DC (VDC) was not toxic. FIG. 20A are immunocytochemistry images showing FB/Dead and live in TCP (tissue culture plate), native, DC and VDC cells. FIG. 20B are immunocytochemistry images showing corneal epithelial in TCP, native, DC and VDC cells. FIG. 20C are immunocytochemistry images showing keratocytes in TCP, native, DC and VDC cells. FIG. 20D are immunocytochemistry images showing endothelial cells in TCP, native, DC and VDC cells. Immunocytochemistry data revealed that VDC allowed maintenance of the phenotype of corneal cells.

FIG. 21 is a line graph showing the proliferation rate of keratocyte induced fibroblast. The vitrified cornea allows for fast proliferation of keratocyte induced fibroblasts as compared to the tissue culture plate and other corneas.

FIG. 22 is a line graph showing the proliferation of epithelial cells. Although the proliferation rate of epithelial cell was much lower than that of TCP, the cornea allowed proliferation of corneal epithelial cells.

FIG. 23 is a line graph showing the proliferation of endothelial cells. The vitrified cornea allowed fast proliferation of endothelial cells, and allowed proliferation of all types of corneal cells.

FIGS. 24A and 24B are images showing the pocket lamellar transplantation model FIG. 24A shows a cartoon of the pocket lamellar transplantation rabbit model, demonstrated that VDC was a potential candidate for use as a stable corneal inlay. FIG. 24B are images showing representative gloss features of the vitrified decellularized cornea in a rabbit recipient. The recipient rabbit eye after transplantation of lamellar vitrified cornea, the gross feature of 1 month, 2 months and 6 months after transplantation proved the vitrified cornea kept its transparency and no haze in the surrounding cornea developed.

FIGS. 25A and 25B are images showing pathological evaluation of VDC. FIG. 25A shows pathological data from one month, 100 μm scale (left), and 50 μm scale (right). FIG. 25B shows pathological data from 6 months 100 μm scale (left), and 50 μm scale (right). Overall, the VDC present indicates its ideal biocompatibility with a rabbit lamellar transplantation model. Through the experiment, there were no immune mediated cells around the decellularized implant (at 30 days post-surgery) and several keratocytes from donor populated around decellularized implant were observed. Donor and implanted cornea started to connect each other with collagen which may be stimulated from donor originated keratocyte. In 180 days post-surgery (6 months), no immune response in the cornea and no keratocyte migration was observed, which may cause the reconstruction of the vitrified decellularized cornea.

FIGS. 26A and 26B are images showing the lithography method and the vitrified decellularized cornea. FIG. 26A is a schematic showing the lithography method, starting with master structures, and finishing with free standing structures. FIG. 26B is an image of the external feature of the shaped vitrified decellularized cornea. After applying the lithography method and the riboflavin crosslinking process, the macrostructure of vitrified decellularized cornea was modified for fitting the corneal contour of each patient.

FIG. 27 a schematic showing the molding method used to produce the catered-VDC. The mold was made using a 3D printer and a 3D OCT (optical coherence tomography) system, and other procedures follow.

FIGS. 28A and 28B are images showing schematic features for animal models. FIG. 28A is an image showing that the partial keratoplasty model is used for evaluating the potential of the vitrified decellularized corneas as a corneal substitute. FIG. 28B is an image showing that the shaped vitrified decellularized cornea is used to evaluate its potential as a corneal inlay.

FIGS. 29A and 29B are derived from 3D and 2D optical coherence tomography. (A) Representative gloss feature, 3D and 2D optical coherence tomography (OCT) images for the mold, the flat and the shaped vitrified decellularized cornea (VDC). (B) Quantitative analysis of curvature for the mold and VDC (n=4).

FIG. 30 illustrates implantation of the vitrified decellularized cornea (VDC) into rabbits with anterior lamellar keratoplasty. External features and the re-epithelial process of the control (n=1) and the VDC applied rabbit corneas (n=3) after the lamellar keratoplasty during 1-month period.

FIGS. 31A and 31B illustrates a pathological examination for the rabbit cornea implanted and the vitrified decellularized cornea (VDC) with anterior lamellar keratoplasty. Images of H&E staining (A) and transmission electron microscopy (B) for the control (n=1) and the vitrified decellularized cornea (VDC) applied rabbit corneas (n=3) after the lamellar keratoplasty. Scale bar for (A): 100 μm.

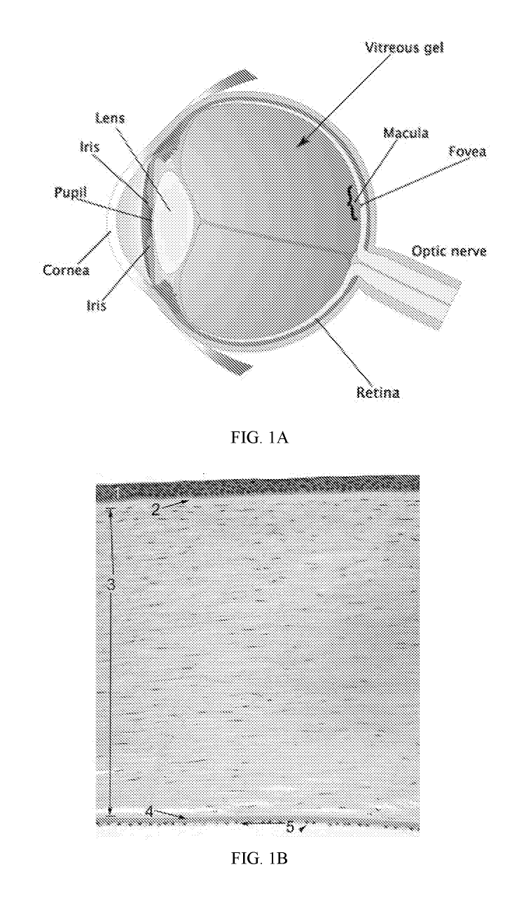

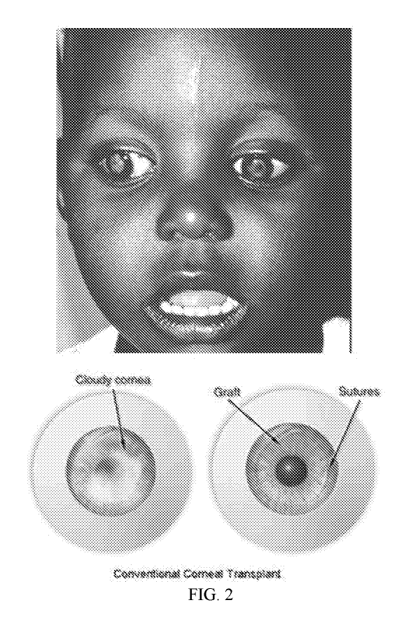

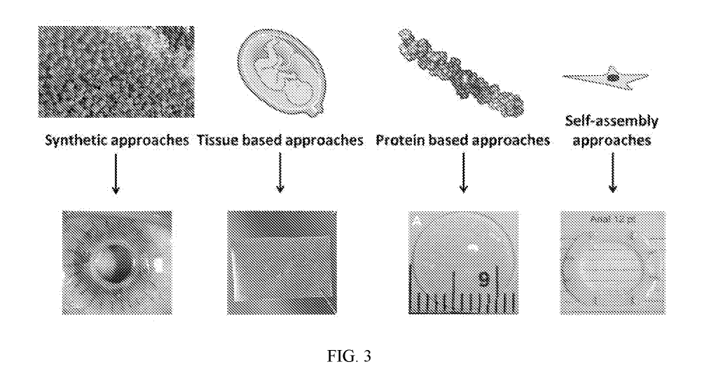

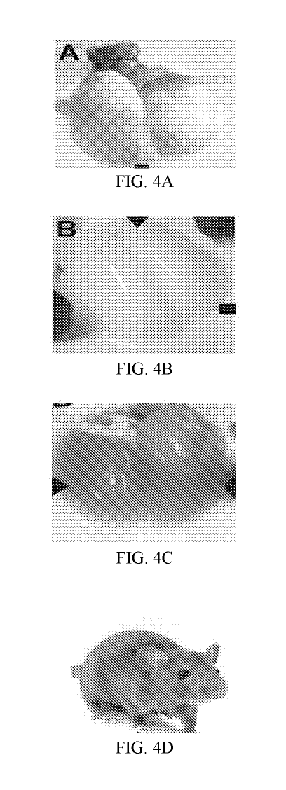

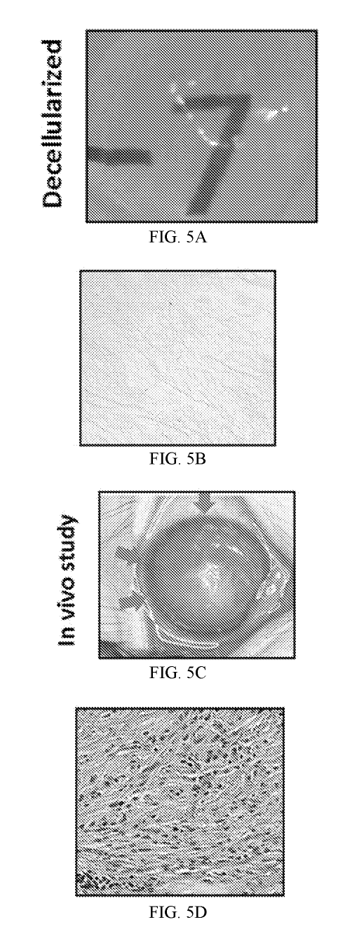

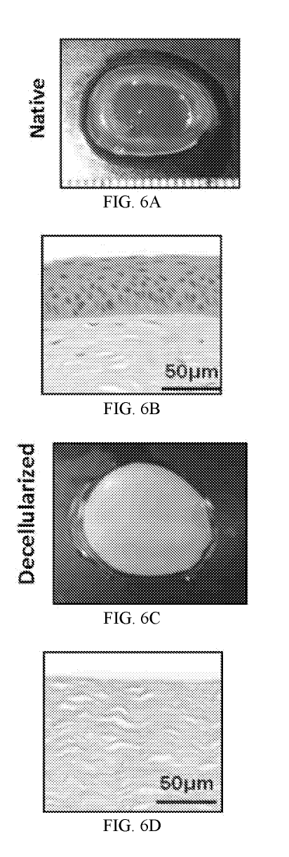

### DETAILED DESCRIPTION OF THE INVENTION

The present invention provides material that may be used for corneal transplantation. In particular, the translational material described herein may be used as a reliable corneal substitute, as well as a stable corneal inlay. The methods described herein (e.g., treatment of animal tissue by decellularization, vitrification, and molding procedures) meet the clinical requirements in terms of optical, biomechanical and biological properties for regenerative medicine. The present invention is based, at least in part, on the discovery that a tissue-based material that undergoes multiple decellularization processes, vitrification (e.g., drying), and riboflavin cross-linking generates a vitrified decellularized corneal inlay that can be used to replace a diseased cornea and manipulate the corneal refractive power, thereby treating patients diagnosed with a corneal disease (e.g., corneal blindness, or corneas with refractive error). The tissue-based material may be highly transparent, biocompatible, stable (e.g., showed ability to control mechanical properties), and showed no remodeling by keratocytes in the cornea of a rabbit.

### Anatomy and Function of the Cornea

The cornea is a highly specialized transparent tissue located at the anterior most surface of the eye. It provides two-thirds of the optical power of the eye, refracting and focusing incident light on the retina, and plays a protective role in the eye by acting as an external barrier to infectious agents. The cornea is composed of three tissue layers: the outer stratified squamous epithelium, the inner endothelium, and the intermediate stroma. The stroma makes up 90% of the corneal thickness and is comprised of a heterodimeric complex of type I and type V collagen fibers, which are arranged in bundles referred to as lamellae. The parallel arrangement of the lamellae as well as the uniform spacing of the fibers, are thought to result in “destructive interference” of incoming light rays, thereby reducing scatter and promoting corneal transparency.

### Corneal Disease and Treatment

Corneal disease affects more than ten million people in the world and is, after cataracts, the second leading cause of blindness. Corneal blindness is currently the 4^{th} cause of blindness. Corneal transplantations (e.g., allergenic corneal transplantations) are currently the standard treatment for restoring vision in many cases. Corneal transplantation and refractive surgery are safe and widely used methods to treat corneal blindness and refractive error respectively. Fortunately, the success rate is generally high. However, a sufficient amount of high quality donors has not been available except in North America. The guarantee of a future with sufficiently high quantity and quality donors may be uncertain due to increases in corneal refractory surgeries and transmissible diseases such as HIV. Many groups have been developing engineered corneas to address these issues. However, all corneas engineered to date have been unable to reach the ideal characteristics of corneal application including transparency, proper curvature, non-toxicity, non-immunogenicity, and proper mechanical and biological properties.

Additionally, refractive error is the most common eye problem, and refractive surgery is a viable option for treatment. However, conventional refractive surgeries are not available for patients who have a thin cornea due to the risk of severe complications including keratoconus. Intraocular lens implantation is an applicable technique for such patients, but a stable biomaterial should be developed to guarantee successful procedures. The biomaterial could serve as a reliable corneal substitute and corneal Inlay to addresses these issues. However, none of the materials at present has the ideal characteristics for these applications including transparency, proper concave shape, biocompatibility and good integration with host tissue. Most importantly, if the corneal inlay is not stable, the visual acuity could not be kept and finally the quality of vision could be deteriorated. Although laser-assisted subepithelial keratectomy (LASEK) and laser in situ keratomileusis (LASIK) are dominantly used in refractive surgery, these surgical methods could be limited for patients with thin corneas due to the potential severe complication, keratoconus. In such cases, intracorneal implantation could be a viable option. However, a reliable corneal inlay is needed to ensure the success of the refractive surgery.

Many groups have been developing engineered corneas to address these issues. Several engineering approaches for corneal reconstruction include: synthetic approaches, tissue based approaches, protein-based approaches and self-assembly approaches. The biosynthetic collagen-based corneal substitute is an example of such an effort. Yet, all engineered corneas to date were unable to reach the ideal characteristics of corneal application including transparency, proper curvature, non-toxicity, non-immunogenicity, and proper mechanical and biological properties. Additionally, the material should not allow remodeling by keratocyte when it is used for intracorneal implantation since the remodeled material could not kept its function, generating acute refractive power.

### Decellularization

Decellularization is the process used in biomedical engineering to isolate the extracellular matrix (ECM) of a tissue from its inhabiting cells, leaving an ECM scaffold of the original tissue, which can be used in artificial organ and tissue regeneration. Organ and tissue transplantation treat a variety of medical problems, ranging from end organ failure to cosmetic surgery. One of the greatest limitations to organ transplantation derives from organ rejection caused by antibodies targeting cell surfaces of the donor organ. Because of unfavorable immune responses, transplant patients suffer a lifetime taking immunosuppressing medication. This process creates a natural biomaterial to act as a scaffold for cell growth, differentiation and tissue development. By recellularizing an ECM scaffold with a patient's own cells, the adverse immune response is eliminated.

With a wide variety of decellularization-inducing treatments available, combinations of physical, chemical, and enzymatic treatments are carefully monitored to ensure that the ECM scaffold maintains the structural and chemical integrity of the original tissue. Scientists can use the acquired ECM scaffold to reproduce a functional organ by introducing progenitor cells, or adult stem cells (ASCs), and allowing them to differentiate within the scaffold to develop into the desired tissue. The produced organ or tissue can be transplanted into a patient. In contrast to cell surface antibodies, the biochemical components of the ECM are conserved between hosts, so the risk of a hostile immune response is minimized. Proper conservation of ECM fibers, growth factors, and other proteins is imperative to the progenitor cells differentiating into the proper adult cells. The success of decellularization varies based on the components and density of the applied tissue and its origin.

Recently, a promising approach using xeno-originated decellularized cornea has emerged. The approach allows remaining a various functional proteins such as integrin that improve biological properties, and keeps its natural construction relatively well to maintain its natural mechanical properties. However, the creation of a decellularized cornea which is transparent and does not contain immunogenic material has not been achieved.

The gentle decellularizing methods may keep the corneal transparency, but may also cause huge immune responses after corneal transplantation. The harsh decellularizing methods may remove the corneal cells and its debris that causes immune responses, but it decreases transparency of the cornea.

### Vitrification

Vitrification is characteristic for amorphous materials or disordered systems and occurs when bonding between elementary particles (atoms, molecules, forming blocks) becomes higher than a certain threshold value. Thermal fluctuations break the bonds; therefore, the lower the temperature, the higher the degree of connectivity. Alternatively, it is a process by which evaporation of water occurs under controlled temperature and humidity (e.g., the corneas may be vitrified in a chamber kept at either 4° C. or 37° C. and about 40% humidity).

### Corneal Keratocyte

Situated between the collagen lamellae in the stroma are the keratocytes, or fibroblasts, which are a population of quiescent, mesenchymal-derived cells of the mature cornea. Corneal keratocytes (corneal fibroblasts) are specialized fibroblasts residing in the stroma of the cornea. This corneal layer, representing about 85-90% of corneal thickness, is built up from highly regular collagenous lamellae and extracellular matrix components. These cells exhibit a slow turnover and are sparsely arranged in the stroma, yet they form an interconnected cellular network with one another through dendritic processes. Keratocytes also contain crystallins; highly expressed proteins that are known to contribute to the transparent nature of the cornea. Keratocytes play the major role in corneal transparency, wound healing, and synthesis of its components. Upon injury, keratocytes are stimulated to either undergo cell death or to lose their quiescence and transition into repair phenotypes. These repair phenotypes can either promote regeneration or they can induce fibrotic scar formation, the latter of which is detrimental to the otherwise transparent cornea. Any glitch in the precisely orchestrated process of healing may cloud the cornea, while excessive keratocyte apoptosis may be a part of the pathological process in the degenerative corneal disorders such as keratoconus. Recently, there has been an interest in the response of keratocytes to injury due to the expansion in development and application of keratorefractive surgeries for correcting vision.

### Glycosaminoglycan (GAGs)

Glycosaminoglycans (GAGs) are the most abundant heteropolysaccharides in the human eye. GAGs are long unbranched polysaccharides consisting of a repeating disaccharide unit. GAGs are highly negatively charged molecules, with an extended conformation that imparts high viscosity to solutions. The repeating unit consists of an amino sugar, along with an uronic sugar or galactose. Glycosaminoglycans are highly polar and attract water. One of the main functions of a class of GAGs, keratan sulfates (KS), is the maintenance of tissue hydration. Within the normal cornea, dermatan sulfate is fully hydrated whereas keratan sulfate is only partially hydrated suggesting that keratan sulfate may behave as a dynamically controlled buffer for hydration. In disease states such as macular corneal dystrophy, in which the level of GAGs such as KS are altered, loss of hydration within the corneal stroma is believed to be the cause of corneal haze, thus supporting the notion that corneal transparency is dependent on proper levels of keratan sulfate. The corneal transparency is due to the uniform distribution of collagen fibrils, which is regulated by proteoglycans. Keratan sulfate GAGs are found in many other tissues besides the cornea, where they are known to regulate macrophage adhesion, form barriers to neurite growth, regulate embryo implantation in the endometrial uterine lining during menstrual cycles, and affect the motility of corneal endothelial cells.

Their biophysical functions depend on their unique properties: the ability to fill a space, to bind and organize water molecules, and to repel negatively charged molecules. Because of high viscosity and low compressibility, they are ideal lubricants in the eyes. On the other hand, their rigidity provides cells with structural integrity and resistance to deformation, and allows cell migration.

Finally, GAGs are a major component of the extracellular matrix (ECM), the “filler” substance existing between cells in an organism. Here they form larger complexes, binding to proteoglycans, to hyaluronan, and to fibrous matrix proteins, such as collagen. They have also been shown to bind with cations (such as sodium, potassium, and calcium) and with water, and their role in regulating the movement of molecules through or within the ECM has also been demonstrated. Individual functions of proteoglycans can be attributed to either the protein core or the attached GAG chain. For all these reasons, GAGs are considered to be the “glue” of the cornea, responsible for providing plasticity and the structural support needed for successful corneal function.

### Riboflavin Crosslinking

Cross-linking of collagen refers to the ability of collagen fibrils to form strong chemical bonds with adjacent fibrils. In the cornea, collagen cross-linking occurs naturally with aging due to an oxidative deamination reaction that takes place within the end chains of the collagen. It has been hypothesized that this natural cross-linkage of collagen explains why keratoectasia (corneal ectasia) often progresses most rapidly in adolescence or early adulthood but tends to stabilize in patients after middle-age. Corneal crosslinking can also be used in combination with other technologies, with the goal of improving the visual results more rapidly. Tiny plastic inserts known as Intacs, which are surgically implanted within the cornea, have been shown to work well with crosslinking. Surface laser vision correction guided by corneal topography has also proven to be a useful technology.

In corneal crosslinking, riboflavin drops are applied to the patient's corneal surface. Once the riboflavin has penetrated through the cornea, UV-A light therapy is applied. This induces collagen crosslinking, which increases the tensile strength of the cornea. Crosslinking with riboflavin and UV-A light has proven to be a first-line treatment for people with eye conditions such as keratoconus, pellucid marginal degeneration and corneal weakness (ectasia) after LASIK.

### Cyclodextrins (Assembly Small Molecules)

Cyclodextrins (sometimes called cycloamyloses) are a family of compounds made up of sugar molecules bound together in a ring (cyclic oligosaccharides). They can form water-soluble complexes with lipophilic drugs, which ‘hide’ in the cavity. Cyclodextrins can be used to form aqueous eye drop solutions with lipophilic drugs, such as steroids and some carbonic anhydrase inhibitors. The cyclodextrins increase the water solubility of the drug, enhance drug absorption into the eye, improve aqueous stability and reduce local irritation. Cyclodextrins are useful excipients in eye drop formulations of various drugs, including steroids of any kind, carbonic anhydrase inhibitors, pilocarpine, cyclosporins, etc. Their use in ophthalmology has already begun and is likely to expand the selection of drugs available as eye drops.

This invention is further illustrated by the following examples which should not be construed as limiting. The contents of all references, patents, and published patent applications cited throughout this application, as well as the figures, are incorporated herein by reference.

### Examples

### Example 1: Materials and Methods

### Vitrified Decellularized Cornea Preparation

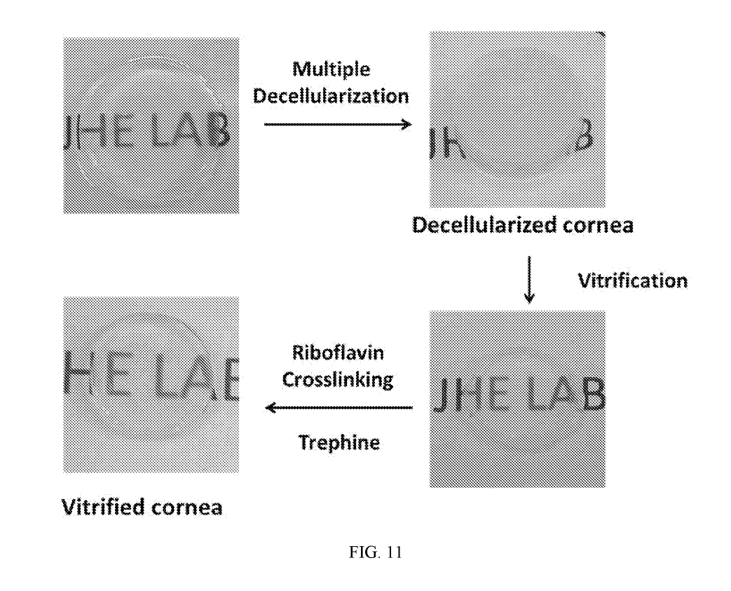

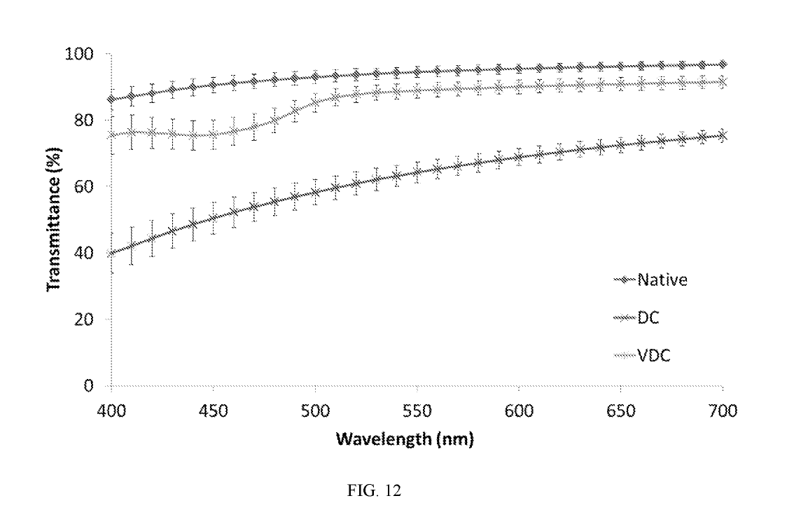

Two procedures, vitrification and riboflavin crosslinking were implanted. The full thickness cornea buttons were prepared using a 12 mm-diameter biopsy punch. After the epithelium was scraped off, the corneas were washed in 5% antibiotic solution in PBS three times. After washing corneal buttons, the native porcine cornea (FIG. 11) was treated with 1% SDS followed by 1% Triton-X for 3 days at room temperature respectively (FIG. 11, decellularized cornea). The corneas were washed in sterile PBS with agitation to remove any remaining chemical agents. Next, the corneas were placed in 10% FBS solution with DMEM for 3 days at 37° C.

After washing the cornea as above, the cornea was vitrified in a chamber kept at either 4° C. or 37° C. and about 40% humility (FIG. 11,). Afterwards, the cornea was immersed in 20% dextrose and 0.1% riboflavin solution overnight and the UV radiation was applied for 3 hours on each side (FIG. 11 vitrified cornea). Following the procedure, the transparency of the cornea was reconstructed microstructurally.

### Material Characterization

The physical and biological properties of VDC were evaluated. The physical properties measured were elastic modulus, tensile strength, material organization and macro and micro-morphology using the indentation method with an Electroforce 3200 testing instrument, the ultimate elongate test with an Instron 5942 system, differential scanning calorimetry with a PerkinElmer DSC 8000 system, the paraffin embedding method with Hematoxylin and Eosin staining and transmission microscopy with a Philips 420 system.

The biological properties of the VDC including biocompatibility, gene expression and corneal epithelial migration rates were tested. Biocompatibility of the material were tested by the Life Technology live/dead assay using keratocytes, gene expression was checked using StepOnePlus Real-Time PCR System with corneal cells and the rate of epithelial cell migration was measured by the Oris Cell Migration Assay test.

### Generating the Shape of the Decellularized Cornea

The vitrified decellularized cornea is shaped with a molding method with a 3D printer and a 3D OCT system. After evaluating the corneal shape of each animal with 3D OCT, information of corneal shape is directly translated to a 3D printer. The 3D printer prints out a couple of molds which fit for the contour and the thickness of animal cornea. The decellularized cornea is vitrified and cross-linked on the surface of the 3D printed mold as per the parameter in the vitrified decellularized cornea preparation.

### In Vivo Transplantation

For evaluating the potential of VDC as a corneal substitute, the partial lamellar keratoplasty model was used. Four experimental and one control (total 5 animals) New Zealand white rabbits are used. All procedures were performed under the general anesthesia with Ketamine (35 mg/kg of body weight) and Xylazine (5 mg/kg of body weight) administered intramuscularly. To minimize the damage of material by nictitating membrane, two horizontal mattress sutures using a 4-0 Vicryl are placed between the free edges of the nictitating membrane to the superior eyelids. The corneas were scored for a depth of about 150 μm using an 8 mm Hessburg-Barron vacuum trephine. After removing the corneal button with an ophthalmic crescent knife, the same size of VDC was inserted on the wounded cornea. The control rabbit did not receive any materials. The material was affixed with 10-0 nylon suture using the interrupted suture method. After surgery, two drops of atropine sulfate were applied to prevent cycloplegia (paralysis of the ciliary muscle of eye which results excessive pain) every day for 3 days. The neomycin, polymyxin B, and dexamethasone ophthalmic ointment (Bausch & Lomb, Tampa, Fla.) was administered to the operated eye once daily for 14 days. Treatment of ocular discharge was done twice daily until the day 7 time point and 3 times a week thereafter until the end of the experiment. An Elizabeth collar was applied to prevent self-trauma until 1 month post-surgery. Ophthalmic examinations were conducted just after the surgery (day 0) and at 1, 2 weeks, 1, 2 and 3 months after surgery. At each time point, an external examination was conducted and the re-epithelialization is evaluated with 0.05% fluorescein under the blue light.

Additionally, in vivo confocal microscopy and optical coherence topography were performed to evaluate the healing process of corneas. The rabbits were euthanized 3 months after transplantation for pathological examination including Hematoxylin and Eosin staining, Masson's trichrome staining, immunohistochemistry and transmission electron microscopy.

### Intra-Stromal Implantation Model

For evaluating the potential of VDC as a corneal inlay, the intra-stromal implantation model is used. The animal number, the breed, the animal group, the anesthetic method follow the above experiment. Under the general anesthesia, the corneal pocket is made in the center of the cornea. Using an IntraLase femtosecond laser system, a 4.7 mm-diamter, 4.9 mm side-cut entry width and 160 μm depth of pocket are made. After lifting the flap, a curved VDC which has a thickness of 50.0 μm and a diameter of 3.8 mm is placed on the central cornea. After, post-operative care follows the above experiment but antibiotic is applied only for 1 week. Ophthalmic examination is performed at after the surgery (day 0) and at 1, 2 weeks, 1, 2 and 3 months after surgery with a slit lamp microscope, an in vivo confocal microscope, a handheld keratometer and an optical coherence topography system. The animals were euthanized 3 months after transplantation for pathological examination as the above experiment.

### Example 2: Physical Characterization of Animal Tissue Based Material

The physical properties of VDC (vitrified decellularized cornea) were evaluated to provide information to develop clinical applications. The porcine tissue based material's mechanical properties were evaluated with that of a native porcine cornea. The physical properties measured were elastic modulus, tensile strength, material organization and macro and micro-morphology. The resulting data was used to optimize further techniques with need-based strategies and are compared with in vivo data to provide insight on corneal tissue.

### Multiple Decellularization

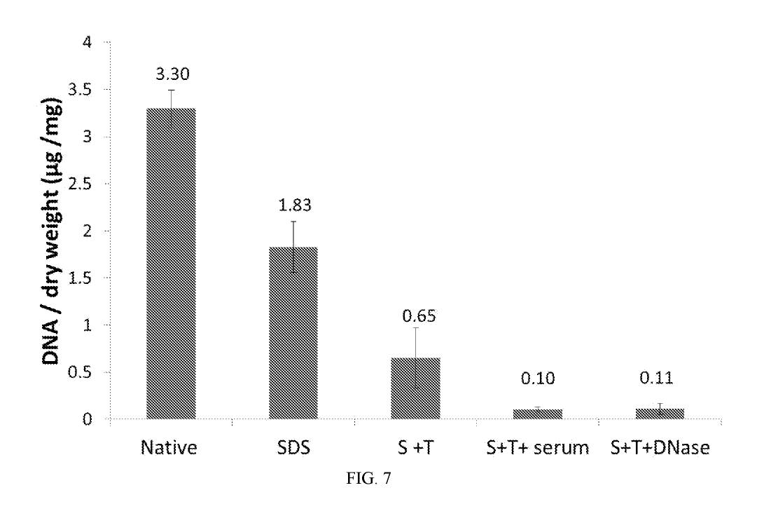

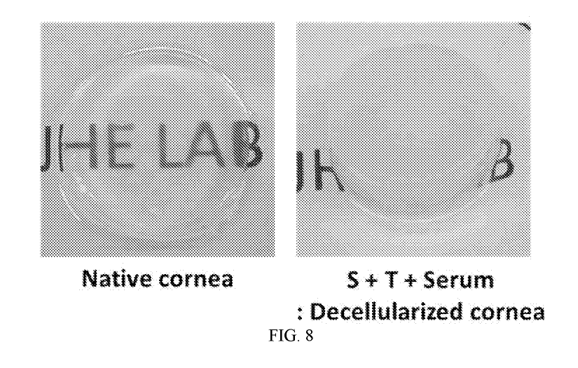

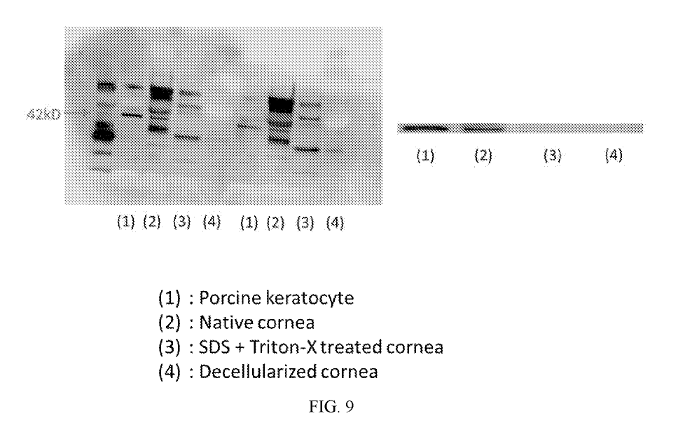

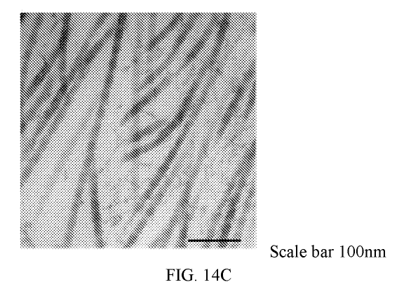

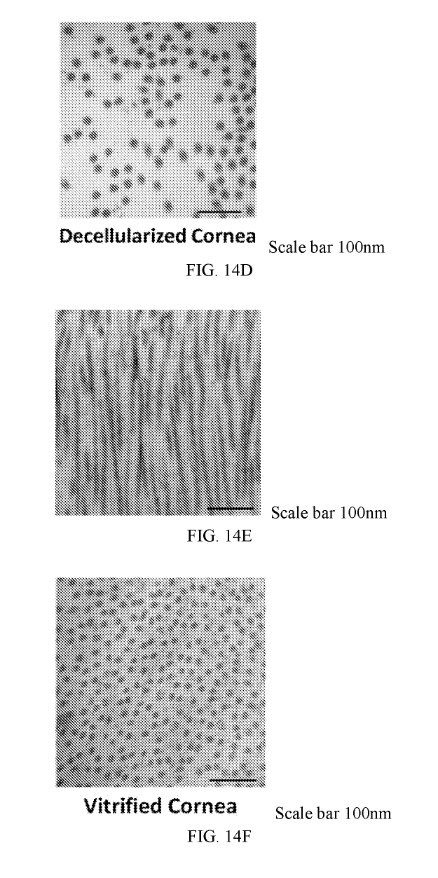

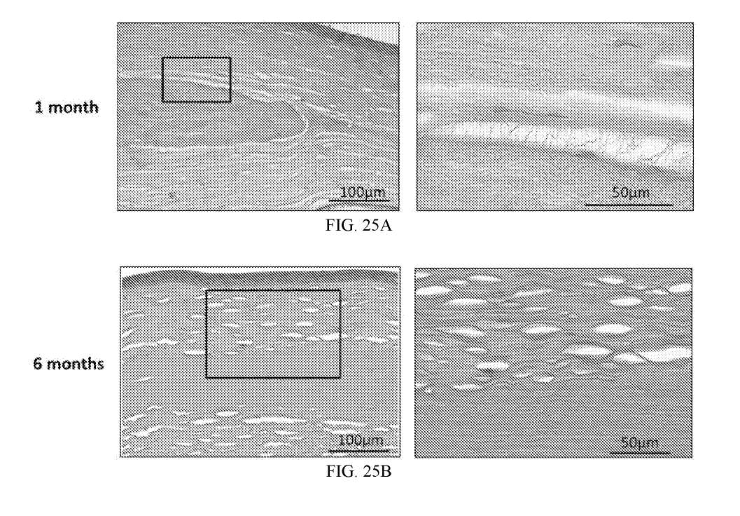

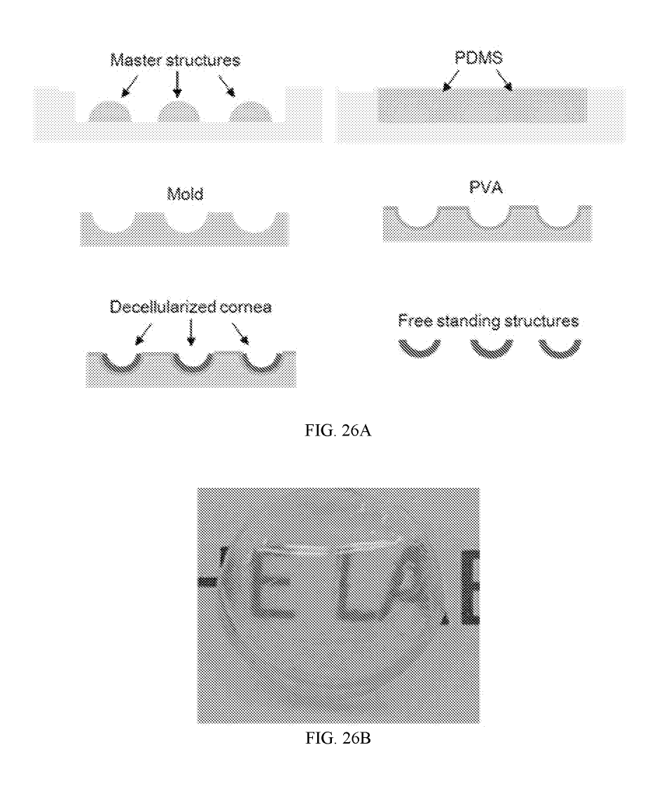

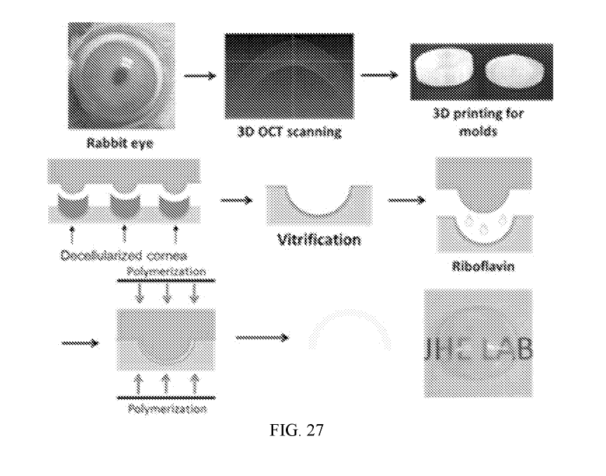

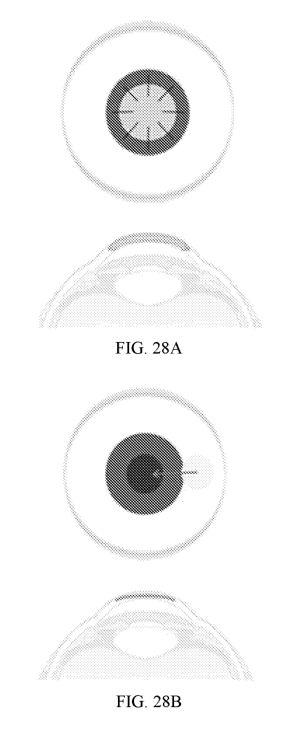

An animal tissue based material with the multiple decellularization procedure was developed, and porcine cornea cells were successfully removed. However, although immunogenic contents (e.g., debris) were minimized (FIG. 7), the structure of decellularized cornea (DC) was altered, which led to a loss in transparency and its concave shape. Using a combination of the novel vitrification process and the conventional riboflavin crosslinking method described herein, a high quality decellularized porcine cornea was produced (vitrified decellularized cornea: VDC) with transparency and reconstructed micro-structure (FIG. 11, and FIGS. 14A-14F). Additionally, with the lithography method using PDMS, the concave macro-structure that fits for the contour of each patient's cornea (FIGS. 26A and 26B) was generated. Moreover, the rabbit study utilizing the pocket method demonstrated VDC did not cause immune response and maintained its transparency up to 6 months post-surgery (FIG. 24B and FIGS. 25A and 25B).

ECM collagen and glycosaminoglycans (GAG) were decreased following decellularization procedure.

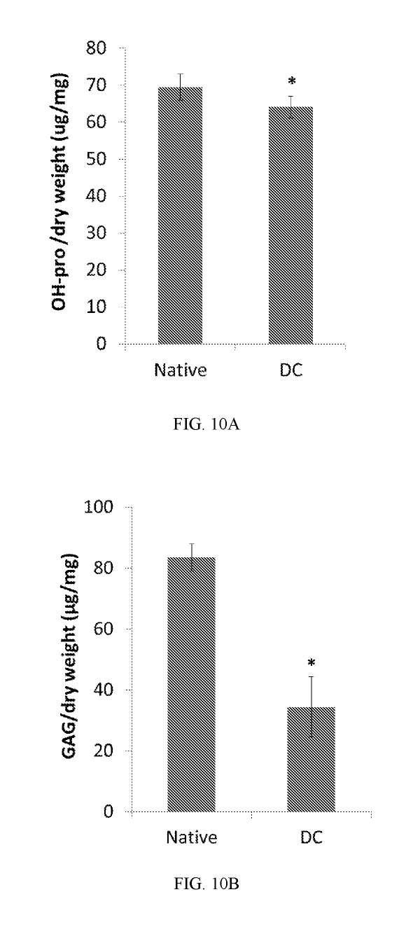

The ECM collagen and glycosaminoglycans were decreased following decellularizing procedures. The OH-pro/dry weight (μg/mg) of decellularized cornea was decreased compared to native cornea (FIG. 10A). The GAG/dry weight (μg/mg) of decellularized cornea was dramatically decreased compared to native cornea (FIG. 10B)

### Microstructure of Vitrified Cornea

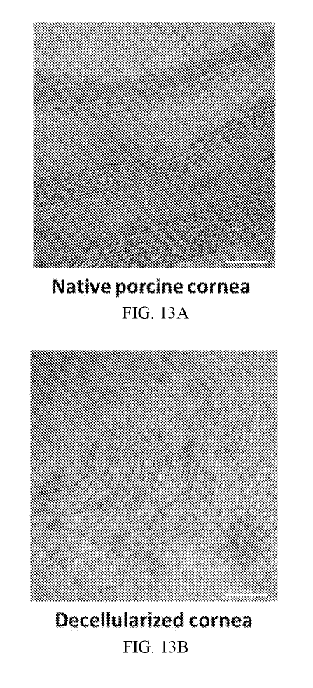

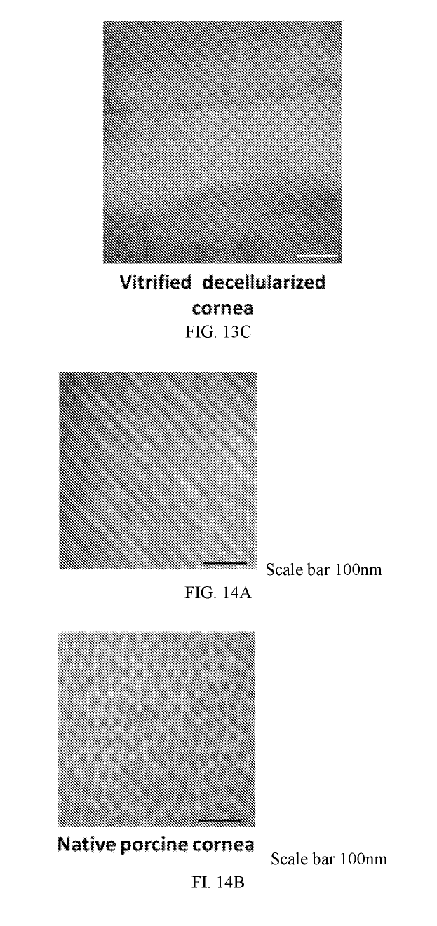

After vitrification and crosslinking, the cornea was reconstructed and the microstructure of the vitrified cornea was evaluated (FIGS. 13A-13C, and FIGS. 14A-14F). TEM images of vitrified cornea showed that the collagen fiber was thinning, and the density of the collagen fibers changed. After decellularizing procedures, the decellularized cornea was recovered to keep transparency and a relatively organized collagen structure.

### Quantitative Measurement of Microstructural Changes

Upon vitrification of the decellularized material, a quantitative measurement of the micro-structural changes was performed. The density of the collagen fiber was evaluated in native, DC and VC samples (FIG. 15A). The number of collagen fibrils increased to almost 250/1 μm² in VC samples as compared to roughly 100/1 μm² in DC samples. The native cornea showed a collagen fiber density of almost 200/1 μm². Additionally, the diameter of the collagen fiber was evaluated in native, DC and VC samples (FIG. 15B). The diameter of the collagen fibers was decreased in VC samples (roughly 30 nm) compared to native corneas (roughly 45 nm). The fiber density and the collagen diameter were not fully reconstructed.

### Macrostructure of Vitrified Cornea

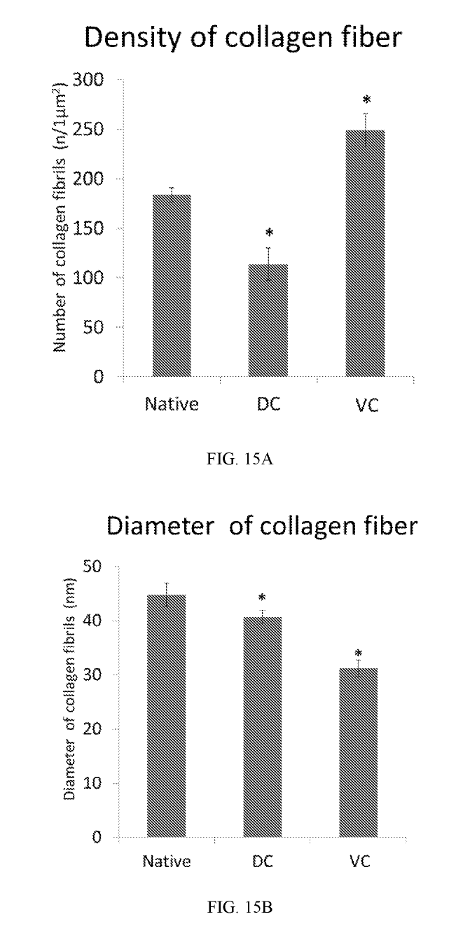

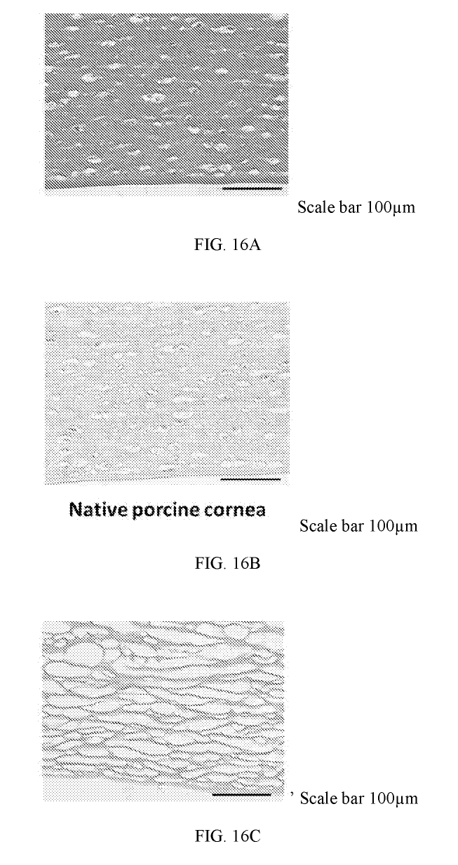

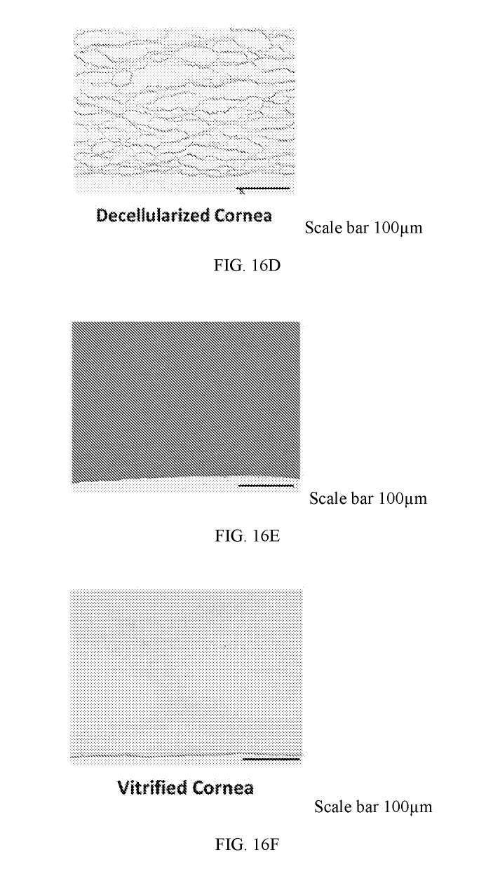

After vitrification and crosslinking, the corneal was reconstructed, and the macrostructure of the vitrified cornea was evaluated (FIGS. 16A-16F). Qualitatively, the GAG content appeared to be increased in defined areas after processing.

### Material Stability of Corneas

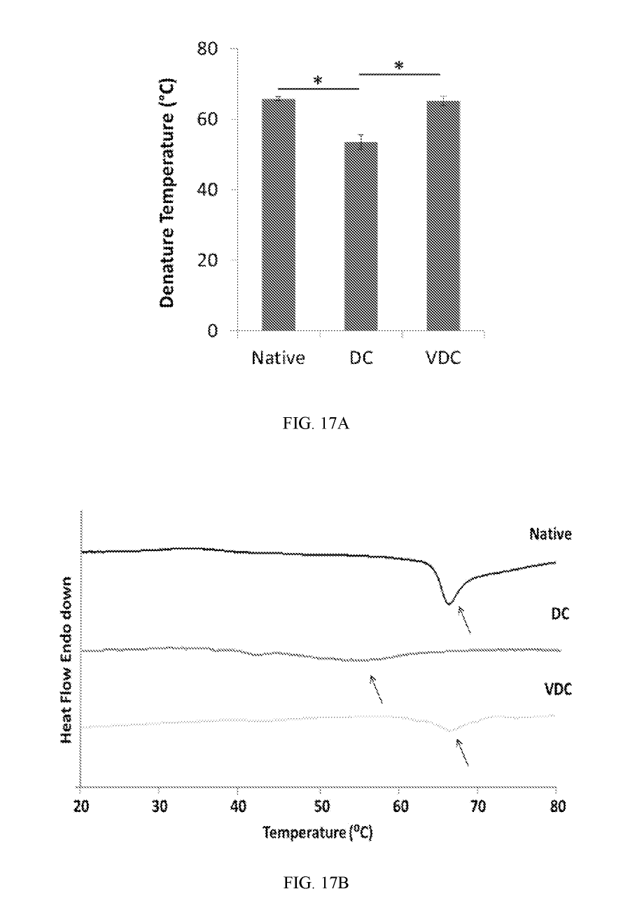

Following vitrification and crosslinking, the material stability of the corneas were evaluated (FIGS. 17A and 17B). The denature temperature of the vitrified cornea, decellularized cornea and native cornea were measured (FIG. 17A). The denaturing temperature of the vitrified cornea was not significantly different compared to that of the native cornea (both roughly 60° C.). The material stability, however was increased compared to the native, DC and VDC samples (FIG. 17B).

### Mechanical Tests for Corneas

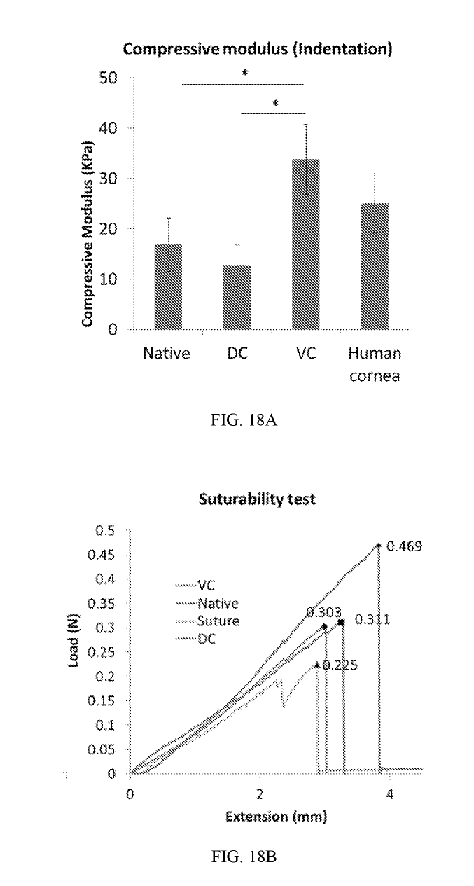

Following vitrification and crosslinking, the mechanical tests on the samples were performed. Using a compressive modulus (indentation) test, the elastic modulus of the cornea samples were evaluated (FIG. 18A). Vitrified cornea samples showed increased compressive modulus (KPa) as compared to native and decellularized corneas. The VC cornea samples, however, were similar to that of the native human cornea (both approximately 25-35 KPa). Additionally, a suturability test was performed on the native cornea, VC, DC and suture (control) samples (FIG. 18B). The vitrified cornea was shown to be a suturable material. Signification was established using ANOVA and Tukey's post-hoc test.

### Example 3: Biological Characterization of Animal Tissue Based Material

The biological properties of VDC (vitrified decellularized cornea) were evaluated to provide information to develop clinical applications. Porcine tissue based material's biological properties are evaluated with that of a native porcine cornea. For measuring the biological properties, biocompatibility, gene expression and corneal epithelial migration rates will be tested. The resulting data is used to optimize further techniques with need-based strategies and will be compared with in vivo data to provide insight on corneal tissue.

### Light Transmittance of VDC

The light transmittance of native cornea, decellularized cornea and vitrified decellularized cornea were evaluated (FIG. 12). From 400 nm to 700 nm the percent transmittance was nearly 100% for native corneas, whereas for decellularized corneas, the percent transmittance was significantly less (e.g., approximately 40% at 400 nm). However, the light transmittance of vitrified decellularized corneas was higher as compared to decellularized corneas, nearly similar to the native cornea, indicating transparency of the cornea.

### Degradation Rate of Corneas in Collagenase Type 1 Solution

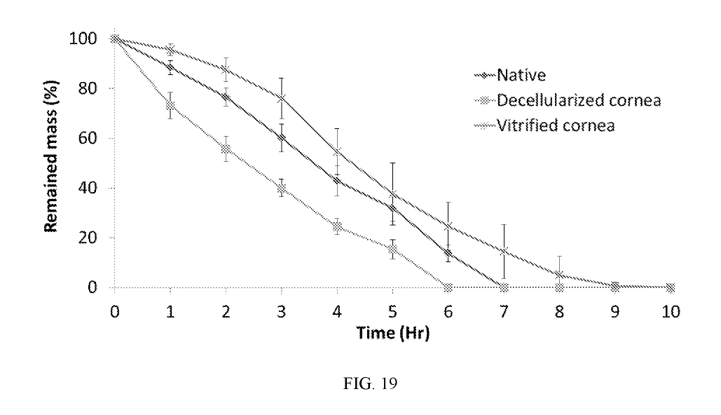

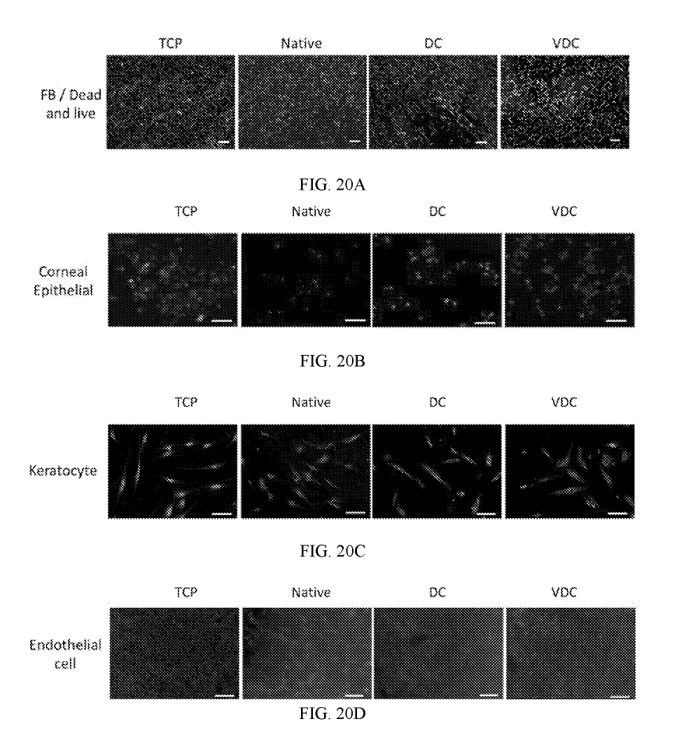

The degradation rate of the cornea samples were evaluated in collagenase type I solution (FIG. 19). The degradation rate of DC was increased after vitrification and crosslinking processes compared to that of the native cornea. The vitrified cornea showed to have a potential to integrate into the host tissue.

### Toxicity of VDC Via Immunocytochemistry

The toxicity of vitrified decellularized corneas were evaluated using immunocytochemistry (FIGS. 20A-20D). The dead and live cell analysis showed that VDC was not cytotoxic. Additionally, the immunocytochemistry data revealed that the VDC allowed for the maintenance of the corneal cell phenotype.

### Proliferation Rate of Keratocyte Induced Fibroblasts, Epithelial Cells and Endothelial Cell

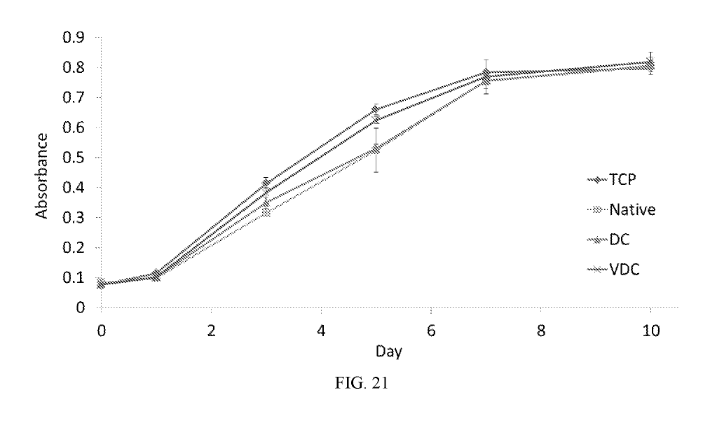

The proliferation rate of keratocyte induced fibroblasts was evaluated in a tissue culture plate (TCP), native corneas, decellularized corneas and vitrified decellularized corneas (FIG. 21). The vitrified cornea allowed for fast proliferation of keratocyte induced fibroblast compared to the tissue culture plate, decellularized cornea and vitrified decellularized cornea.

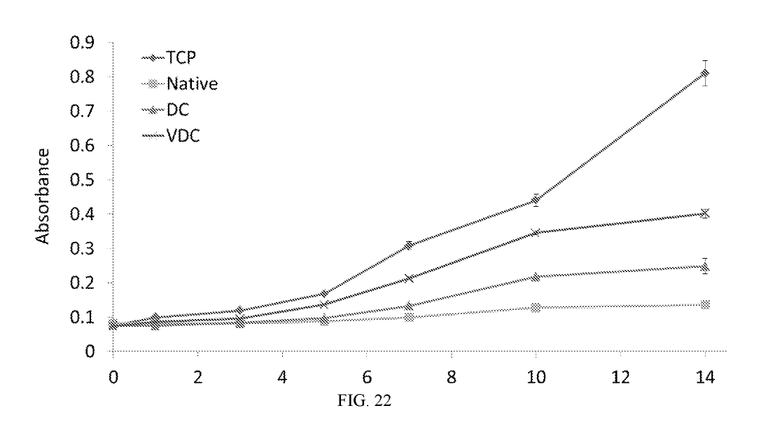

The proliferation rate of epithelial cells was evaluated in a tissue culture plate, native corneas, decellularized corneas and vitrified decellularized corneas (FIG. 22). Although the proliferation rate of the epithelial cells was much lower than that of TCP, the cornea allowed for proliferation of corneal epithelial cells.

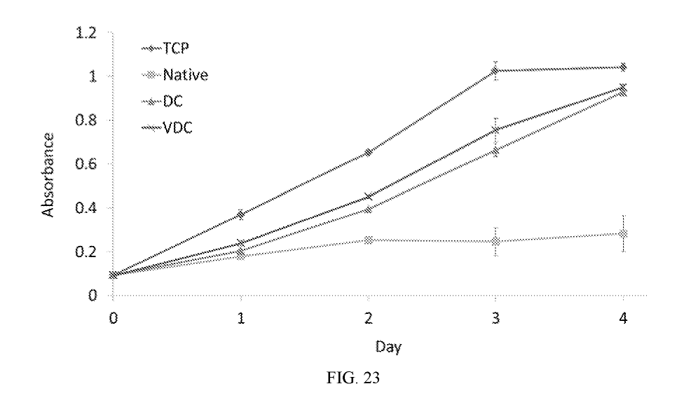

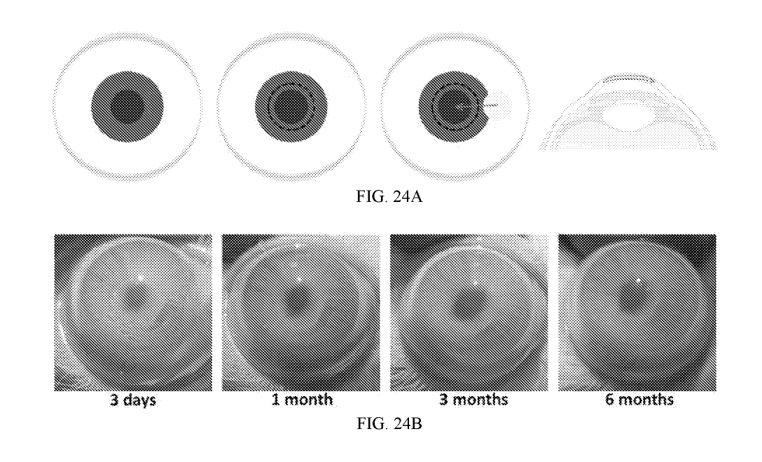

The proliferation rate of endothelial cells was evaluated in a tissue culture plate, native corneas, decellularized corneas and vitrified decellularized corneas (FIG. 23). The vitrified cornea allowed for fast proliferation of endothelial cells. Overall, the vitrified decellularized corneas allowed for proliferation of all types of corneal cells.

### Pocket Lamellar Transplantation Model (FIGS. 24A and 24B)

The pocket lamellar transplantation model was used to evaluate the vitrified decellularized cornea in a rabbit recipient (FIGS. 24A and 24B). FIGS. 24A and 24B are images showing the pocket lamellar transplantation model. FIG. 24B are images showing representative gloss features of the vitrified decellularized cornea in a rabbit recipient. The recipient rabbit eye after transplantation of lamellar vitrified cornea, the gross feature of 1 month, 2 months and 6 months after transplantation proved the vitrified cornea kept transparency and no haze in the surrounding cornea.

### Pathological Evaluation

A pathological evaluation was performed on the vitrified decellularized cornea using a rabbit lamellar transplantation model (FIGS. 25A and 25B). FIG. 25A shows pathological data from one month post-surgery, 100 μm scale (left), and 50 μm scale (right). FIG. 25B shows pathological data from 6 months post-surgery 100 μm scale (left), and 50 μm scale (right). Overall, the VDC present indicated its ideal biocompatibility with a rabbit lamellar transplantation model. Through the experiment, there were no immune mediated cells around the decellularized implant (at 30 days post-surgery) and several keratocytes from donor populated around decellularized implant were observed. Donor and implanted cornea started to connect each other with collagen which may be stimulated from donor originated keratocyte. In 180 days post-surgery (6 months), no immune response in the cornea and no keratocyte migration was observed, which may cause the reconstruction of the vitrified decellularized cornea.

### Example 4: Modification of Animal Tissue Based Material to Desired Shape

The specific concaved shape of cornea generates the refractive power to assure the visual acuity. Although, the decellularized cornea did not maintain its concave shape after processing, the shape is freely modified of the material with a molding method, using a 3D printer and a 3D optical coherence tomography (OCT) system. The method to freely change the corneal shapes restores the refractive function of the animal based material and provides a tool to manipulate the refractive rate of patient's cornea with the corneal inlay. Additionally, this technique allows for the production of patient-catered cornea, and the shape of the scaffold may be modified as clinical needs that may be able to correct the refractive power of the cornea.

### Example 5: In Vivo Translational Applications of the Animal Tissue Based Material as a Corneal Substitute and Corneal Inlay

The shaped-modified, tissue-based material is applied to two animal models: the partial lamellar keratoplasty model and the corneal intrastromal transplantation model. The shaped-modified material is applied as a corneal replacement as well as manipulation of the corneal refraction. The two models are evaluated for the corneal reconstructive potential of this material. Animals are evaluated using clinical observation in the same manner as in the ophthalmic clinic. In addition, various pathological techniques are used. Specific attention is paid to determine the fate of the material after implantation and the relationship between the in vivo results and characteristic properties of material. The experiments generate data used to move technology towards advanced preclinical studies.

### Example 6: Assembly of Vitrified Decellularized Cornea is Augmented by Addition of Additives

The vitrified decellularized cornea is further incubated with an additive that will augment the assembly of the sample. Without being bound by theory, the additive may be a small molecule (e.g., cyclodextrin). Additionally, the small molecule may be an acid (substituted with a hydroxyl moiety).

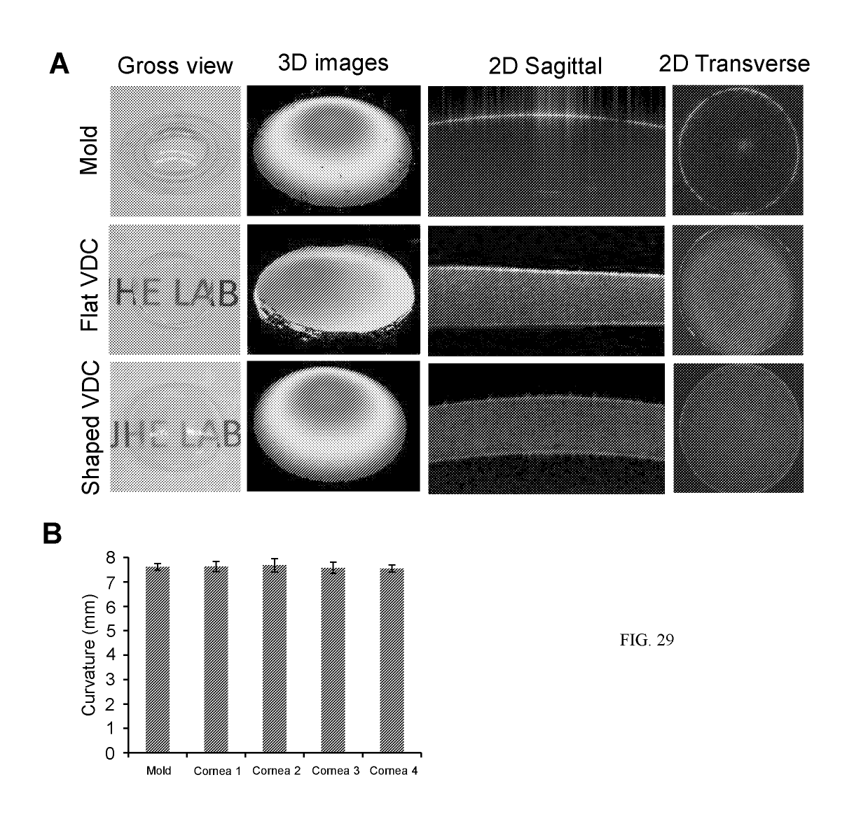

### Example 7: Optical Coherence Tomography and Curvature Analysis (FIG. 29)

The home-built OCT imaging system consists of a swept source OEM engine (AXSUN, central wavelength λ0: 1060 nm, sweeping rate: 100 kHz, scan range: 3.7 mm in air), a balanced photo-detector and a digitizer with a sampling rate of up to 500 MSPS with 12-bit resolution, a Camera Link DAQ Board, and a Camera Link frame grabber (PCIe-1433, National Instruments). For the optical scanning head, 2D galvanometer mirrors (GVS002, Thorlabs) and OCT scan lens with 36 mm effective focal length (LSM03-BB, Thorlabs) were used. The workstation (Precision T7500, Dell) with general-purpose computing on graphics processing units (GPGPU, GeForce GTX980, Nvidia) processed the sampled spectral data and reconstructed the 3D OCT image. The parallel processing (CUDA, Nvidia) of the GPGPU significantly reduced the signal processing time including FFT (Fast Fourier Transform). Finally, 512×512×1024 volumetric OCT images were reconstructed and 10 duplicated 3D images were averaged to increase SNR (Signal-to-Noise Ratio). Based on the reinforced 3D OCT images, canny edge detection algorithm was applied in order to extract the curvature information of the shaped reconstructed cornea as well as the mold. Then, the gray-scale image was converted into a binary image with a specific threshold value to extract the surface line of the cornea and the mold. The surface binary image was rescaled in accordance with the physical scanning size. Using this final image, the mean value of curvature was calculated by measuring 6 (3 by 3) different positions of the cornea.

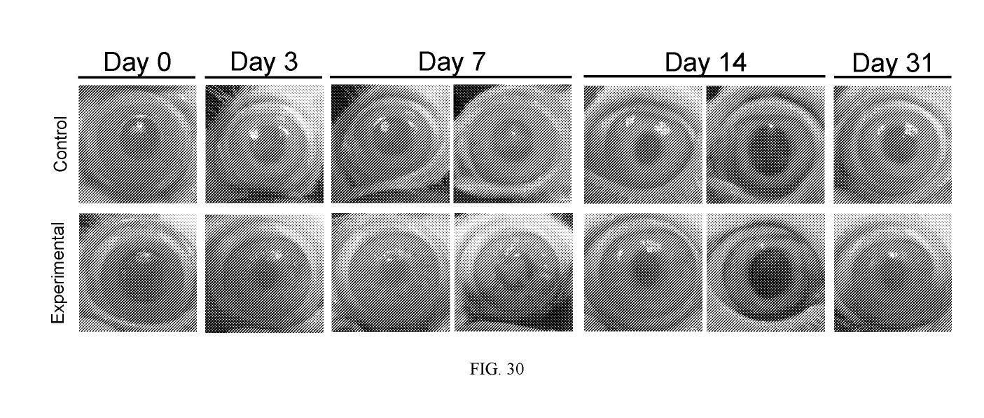

### Example 8: Surgical Procedures for Interlamellar and Anterior Lamellar Keratoplasty (FIG. 30)

To evaluate the clinical potency of VDC as a corneal substitute, a pilot study was carried out using the anterior lamellar keratectomy model with 4 rabbits. Rabbits were randomly divided two groups: 3 rabbits for the VDC group and 1 animal for the untreated negative control. Using 6 mm Hessburg-Barron vacuum trephine (Barron Precision Instruments LLC, Grand Blanc, Mich.) and a crescent knife (LaserEdge, Bausch&Lomb, Rochester, N.Y.), approximately 125 μm of anterior corneal tissue was removed from a randomly chosen eye of each rabbit. After inducing injuries, rabbits in VDC group received a shaped V (approximately 125 μm thickness, 6.25 mm diameter and 7.5 mm curvature) affixed using 12-14 interrupted 10-0 nylon sutures. The negative control group was similarly operated on, but did not receive any material and rather, was allowed to heal naturally. A mixture of steroid and antibiotic ointment (Pred-G, Allergan, Irvine, Calif.) was applied for 14 days as a post-operative treatment. Gross observations, including ophthalmomicroscopy and fluorescein staining were performed at day 3, 7, 14 and 1 month after surgery. In addition, In vivo pachymetry were carried out to calculate the thickness of the animal cornea before sacrificing using a pachymeter (Corneo-Gage Plus™, Sonogage, Cleveland, Ohio). Corneas were harvested after 30 days post-surgery for pathological examinations. Methods for pathological examination were described above.

### Example 9: Evaluation of Macroscopic Structural Reconstruction

Using an OCT system, the regenerated concave shape of VDC regarding curvature of the rabbit cornea was evaluated. The 3D OCT image revealed the shaped VDC had a similar architecture with the mold. In addition, VDC presented a smooth surface in 2D OCT image (FIG. 29 A). In the curvature analysis, the curvature of the reconstructed cornea (7.613±0.136 mm) was identical with that of the mold (7.615±0.138 mm, FIG. 29B).

### Example 10: Anterior Lamellar Keratoplasty Model

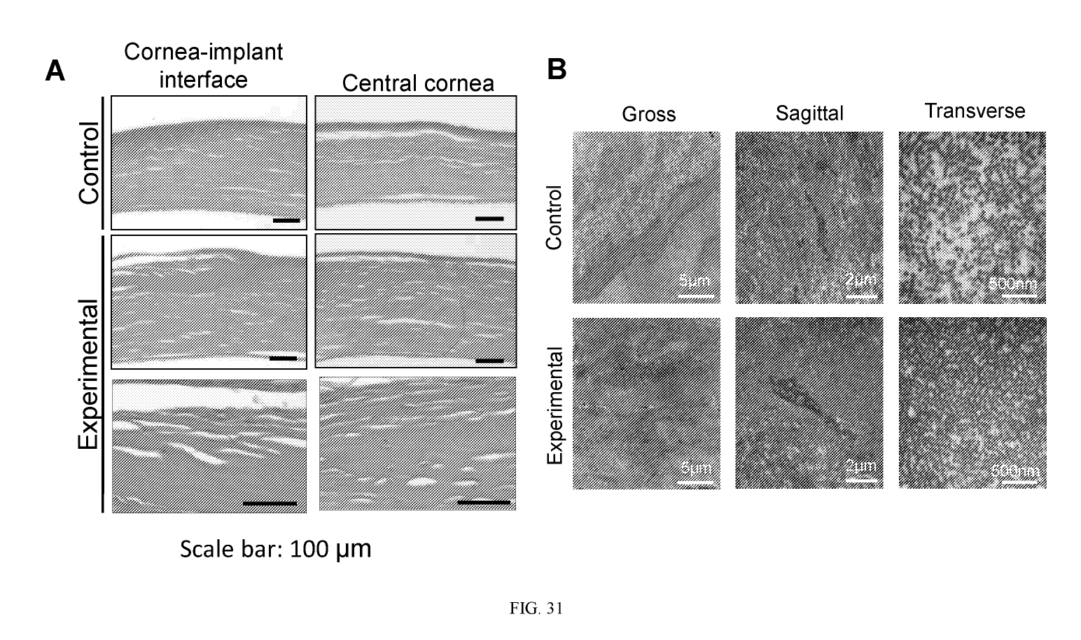

To evaluate the potency of the shaped VDC as a corneal substitute, a rabbit partial keratectomy model was conducted. The re-epithelialization in the VDC implanted cornea was completed before 14 days post-surgery whereas that on the control cornea was done before 7 days post-surgery. Corneal neovascularization, graft degradation, immune rejection and other complications were not observed during the study. The initial thickness of corneas that received VDC and the control cornea was 353.7±10.3 μm and 360 μm respectively. A month later, VDC treated corneas kept their thickness (360.8±9.5) with VDC. However, the control cornea was limited in its ability to regenerate its thickness (275 μm). Although slight corneal haze was found in the VDC implanted group during 30-day period, corneal haze in the experimental group was not as serious as the control cornea (FIG. 30). The pathological examination with H&E staining presented that the VDC implanted cornea allowed cornea epithelial cells as well as keratocytes migration on and into VDC respectively (FIG. 31A). In addition, the migrated keratocytes remodeled the collagen structure of the VDC (FIG. 31A). In ultrastructural evaluation, the VDC treated cornea had not fully integrated with host tissue until 1 month post-surgery. Some gaps between VDC and host cornea were found in the interface. In addition, some keratocytes were found in the implanted VDC. The collagen density of implanted VDC was higher than that of the reconstructed control cornea (FIG. 31B).

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for treating a corneal disease in a subject, the method comprising:
obtaining a tissue-derived scaffold;
decellularizing the tissue-derived scaffold;
vitrifying the tissue-derived scaffold;
cross-linking the tissue-derived scaffold; and
generating a vitrified decellularized corneal inlay, thereby treating corneal disease in the subject.

2. The method of claim 1, further comprising:
molding the vitrified decellularized corneal inlay to produce a modified-shaped cornea for treatment in said subject.

3. The method of claim 1, wherein vitrifying comprises controlled temperature and humidity.

4. The method of claim 3, wherein the temperature is between 4° C. to 37° C.

5. The method of claim 3, wherein the humidity is 40%.

6. The method of claim 1, wherein the cross-linking is riboflavin cross-linking.

7. The method of claim 1, wherein the vitrified decellularized corneal inlay is used to treat corneal blindness.

8. The method of claim 2, wherein the molded, vitrified decellularized corneal inlay is used to correct refractive error.

9. The method of claim 2, wherein the molding is performed using a 3D printer and 3D OCT (optical coherence tomography) system.

10. The method of claim 1, further comprising addition of additives.

11. The method of claim 10, wherein the additives comprise small molecules.

12. The method of claim 11, wherein the small molecule is cylcodextrin.

13. The method of claim 1, wherein the tissue-derived scaffold is from a bladder.

14. The method of claim 1, wherein the transparency of the tissue-based scaffold is preserved.
